(19) United States
(12) Patent Application Publication
(10) Pub. No.: US 2017/0283287 A1
(43) Pub. Date: Oct. 5, 2017

(54) METHOD OF AND APPARATUS FOR OXIDATIVE TREATMENT OF LIQUID, GASEOUS, AND/OR SOLID PHASE

(71) Applicant: Klaus NONNENMACHER, Tübingen (DE)

(72) Inventor: Klaus NONNENMACHER, Tuebingen (DE)

(21) Appl. No.: 15/033,825

(22) PCT Filed: Nov. 11, 2014
(86) PCT No.: PCT/DE2014/000573

§ 371 Date: May 25, 2016

(30) Foreign Application Priority Data
Nov. 20, 2013 (DE) 10 2013 019 339.7

(51) Int. Cl. C02F 1/78; F28C 1/02; A61L 2/18; F28F 25/10; B01D 53/34; C02F 1/72

(52) U.S. Cl. CPC C02F 1/78; B01D 53/34; C02F 1/722; A61L 2/183; F28F 25/10; F28C 1/02

(57) ABSTRACT

The invention relates to a method and a device (100) which are provided for the oxidative treatment of a liquid phase and/or a gas phase and/or a solid phase. According to the invention, ozone and at least one component, which is provided by the ozonization of at least one olefin, is used for the treatment. The method and the device can, for example, be used for waste water treatment.

The present invention relates to a method and apparatus for the oxidative treatment of a liquid phase and/or a gas phase and/or a solid phase. Using the method, for example, contaminated water can be processed.

An oxidative treatment of organic contaminated water is a substantial component of water treatment, for example in connection with a waste-water purification. In addition to biological and physical purification processes, the oxidative treatment of water serves for removing contaminating substances in the water. The use of water leads to the most varied contaminations. Examples of this are pesticides, herbicides, fire-prevention agents, softeners or pharmaceuticals. Further sources of contaminations are swimming pools, paper factories or also the chlorination of drinking water. Thus for example different halogenated organic hydrocarbons enter the water circulation as trace pollutants. Important representatives of these harmful substances are grouped together as AOX substances (adsorbable organically bound halogens) that include chlorine, bromine and iodine compounds. Such substances are often hazardous to health. Other contaminating substances in water are bacteria, viruses, fungi, fungus spores or constituents thereof that can also give rise to a health hazard. For removal of these contaminations a costly water purification and processing is necessary. The integration of oxidative treatment processes into the procedures at water treatment works is an important component.

An oxidative treatment with ozone is known for the decomposition of harmful substances and for disinfection or sterilization of water. In this case ozone acts as an oxidizing agent. In combination for example with UV radiation or with hydrogen peroxide (H₂O₂), the efficiency of the oxidative process can be further increased (Hoigné, Jürg (1998) “Chemistry of aqueous Ozone and Transmission of Pollutants by Ozonation and advanced oxidation processes, in: Hrubec, Ji\check{r}i (1998): Quality and Treatment of Drinking Water II. Berlin, Heidelberg: Springer (The Handbook of Environmental Chemistry, Part C, 5/5 C), pages 83-141).

Ozone is generally produced technically in gaseous form. It dissolves physically in water and does not react with the water. The hydroxyl radical .OH is in particular responsible for the oxidative action of the ozone. A preliminary stage (precursor) of the hydroxyl radical is the hydroperoxide anion HO₂⁻ that in the event of ozone decomposition is produced according to the following reaction equation:

O₃+OH⁻→HO₂⁻+O₂ (G1)

The conversion rate is only moderate, even at higher pH values. The half-life at pH 8 is for example approximately 2.8 hours.

The dissociation of hydrogen peroxide also results in the production of the hydroperoxide anion:

H₂O₂ [symbol] HO₂⁻+H⁺ (G2)

The pKₐ value of hydrogen peroxide is 11.6, so that at higher pH values the balance is shifted toward HO₂⁻. Nevertheless, the precursor formation remains moderate.

By adding hydrogen peroxide in ozone water, that is to say in water with dissolved ozone, a further source for the precursor can be provided. To summarize, the following reaction with ozone and hydrogen peroxide is produced:

2O₃+H₂O₂→2.OH+3O₂ (G3)

Also this overall reaction only proceeds moderately, so that an oxidative water treatment with ozone and hydrogen peroxide often does not achieve satisfactory results.

Formation of hydroxyl radicals is also possible through the ozonide anion O₃⁻ produced by ionizing aromatic compounds according to the following equation:

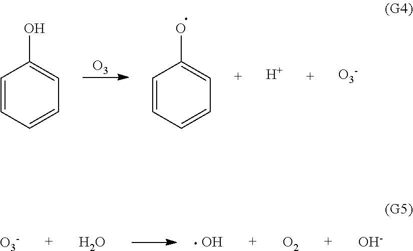

Studies show that in an organic DOM matrix (DOM=dissolved organic matter), in certain cases an unexpectedly high decomposition rate of PCBA (4-chlorobenzoic acid) that is an intermediate product in the bacteriological decomposition of herbicide, can be achieved with a combined ozone and hydrogen peroxide treatment (von Sonntag, Clemens; von Gunten, Urs (2012) Chemistry of Ozone in Water and Waste Water Treatment. London: IWA Publishing). In this connection the organic matrix may be aliphatic or a mixture of aromatic and aliphatic substances. In this case the proportion of the matrix that in the pure ionization favors a hydroxyl radical formation, is merely hydroxylated (R—COOH), but not decomposed completely to CO₂. Therefore, the content of dissolved organic structure (DOC=dissolved organic carbon) is not significantly reduced. Furthermore, however, the peroxide DOM matrix (R—COOH) generates hydroxyl radicals. These persist and influence the stability of the dissolved ozone in the water. Such a hydroxyl radical formation is actually desirable. However, the water treatment should provide a complete removal of the dissolved organic carbons.

On the other hand, the object of the present invention is to provide a new method and a corresponding apparatus for oxidative treatment of contaminated water, wherein the purifying and disinfecting action of the oxidative treatment is improved and more effective. At the same time, it should be possible to implement the method in practice with relatively little effort and low costs. A further feature of the invention is treating exhaust gases or exhaust air or solid materials that are contaminated for example with compounds having strong odors or other impurities or contaminants.

This object is achieved by a method of the oxidative treatment of a liquid phase and/or a gas phase and/or a solid phase as set out in claim 1. Preferred embodiments of this method as well as an apparatus for the oxidative treatment of a liquid phase and/or a gas phase and/or a solid phase as well as an evaporative cooler are the subject of further claims.

The method according to the invention is based on the fact that the liquids to be treated, for example contaminated water, or the gases to be treated, for example exhaust gases, or the solid materials to be treated are subjected to an oxidative combined treatment where on the one hand ozone and on the other hand a component resulting from the ionization of an olefin are used for the treatment. This component, which is also designated below in generalized terms as an ozonide component, is preferably an organic ozonide and/or an organic peroxide and/or mixtures thereof that are provided for the treatment. As a result, in all three cases a purifying and/or disinfecting action is achieved. The addition of the ozone and the addition of the ozonized olefin or optionally of precursor molecules thereof can take place simultaneously or in principle in any order. In principle it is also possible to add the necessary substances in liquid form and/or gaseous form and/or in vapor form and/or in the form of a spray mist. This method of oxidative treatment is suitable for treating liquid phases, gas phases and solid phases (solid materials), and the method is preferably carried out in the presence of water. In this case the water can be added for example in liquid form, gaseous form and/or in the form of an aerosol. Moistening (misting) is provided for example for treatment of gases and for treatment of solid materials. The radical reactions triggered during the method according to the invention always end in principle in an aqueous phase, so that in all three cases (liquid phase, gas phase, solid phase) the chemical reactions are comparable. The ozonide component, the ozone and the water react so that hydroxyl radicals are produced that are the starting point for a chain reaction. In the chain reaction, organic constituents of the phase to be treated react unselectively up to their maximum oxidation stage (CO₂). In this case hydroxyl radicals are again formed as long as organic substance is present.

The crux of the invention is the combined treatment of a liquid, gas, and/or solid phase with ozone with at least one constituent resulting from the ionization of at least one olefin. In this connection the expression “olefin” (or also “alkene”) should be understood to mean generally unsaturated hydrocarbon compounds. These compounds can be both linear and also cyclic. In this case it is essential that the compounds have at least one carbon-carbon double bond. Thus these are unsaturated hydrocarbon compounds or, in other words, an organic substance with an unsaturated hydrocarbon structure. The at least one olefin preferably involves one or more unsaturated fatty acids. For the purposes of the invention, however, other compounds can also be used, for example pure hydrocarbons that have at least one carbon-carbon double bond.

The component resulting from the ionization of at least one olefin is advantageously produced in a separate process whereby an olefin is treated with ozone and is provided for the oxidative treatment. The ionization of the olefins can take place in the presence of water (protic) or in the absence of water (aprotic) whereby primarily organic peroxides are formed under protic conditions and primarily stable organic ozonides are formed under aprotic conditions. The reaction conditions for the ionization of the olefins are preferably chosen as a function of the dew point of the carrier gas used. If for example dry ozone gas is used as carrier gas, the ionization of the olefins can preferably be carried out aprotically. Depending on the reaction conditions, different products can be produced by the ionization. Monomeric and/or polymeric ozonides, for example 1,2,4-trioxolane, are formed for example under aprotic conditions. In particular when protic solvents are used in the ionization reaction, peroxide compositions, in particular organic hydroperoxides or mixtures of the substances, can be formed, for example polymeric or dimeric peroxides, such as for example 1,2,4,5-tetroxane.

Thus, depending on reaction conditions, due to the ionization of the olefins organic ozonide and/or organic peroxide and/or hydroperoxide or mixtures thereof are provided that are used for the method according to the invention. The resulting substance, which is generally designated below as an ozonide substance, can be introduced into the water as an oily or creamy substance. On contact with the water, the organic ozonide decomposes into aldehyde and carboxylic acid fractions. In this case hydrogen peroxide or the hydroperoxide anion that is a precursor of the hydroxyl radical is formed. Therefore, in the presence of ozone, due to the contact of the ozonide with water the hydroxyl radical is ultimately formed that is then available for the oxidative processes for decomposition of the harmful substances. Furthermore, the hydrogen peroxide causes a mineralization of the fractions of the ozonide, that is to say the carboxylic acid and the aldehyde, in carbon dioxide and water.

The component produced by ionizing the olefins is advantageously provided in adsorbed form for the process, for example by adsorption on a packing of pebbles or on other surfaces, for example on metal oxide or ceramic materials or on glass. Materials that are generally suitable for this are in particular pebbles, glass bodies, hollow glass bodies, ceramic, for example kaolin, synthetic materials, textiles or metals, for example in the form of nonwovens, honeycombs, tubes, balls, hollow bodies or bulk materials. The packing advantageously provides a fatty, i.e. hydrophobic surface for the adsorption of the ozonide components. In particular, for the oxidative treatment of water or of gases a packing is provided for an adsorption of the constituent with the ozonized olefins that preferably takes place downstream of a metering point for the ozonide component. A corresponding packing can be provided for example in a tube reactor and/or on the bottom of a tank. The effectiveness of the treatment can be increased by oxidic surfaces in these materials.

The method according to the invention is suitable in particular for oxidative treatment of contaminated water as liquid phase, in particular organically contaminated water. The main focus of the application of the method according to the invention is treating waste water. In principle this method can also be used for oxidative treatment of other liquids, in particular aqueous liquids. For oxidative treatment the water is ozonized, that is to say it is admixed with ozone. In general the ozone is dissolved substantially physically in the water.

Organically contaminated water can be reliably processed by this method, so that the water can for example be fed back to a natural body of water or can be used for the provision of drinking water. With the method according to the invention it is possible above to reliably eliminate trace pollutants that can accumulate in the water circulation. Furthermore, disinfection of the water, in particular for removal of bacteriological contaminants, is achieved by the oxidative treatment according to the invention. Studies by the inventor have shown that with this combined oxidative treatment an unexpectedly high purifying effect is achieved. This is manifested for example in that the ozone consumption for the decomposition of a specific quantity of organic substance falls by approximately a factor of 10. The special purifying effect of the combined treatment according to the invention may be explained as follows. By treating the ozonized water, for example with an ozonized fatty acid as olefin, the ozone (O₃) physically dissolved in the water is ultimately converted to hydroxyl radicals. The hydroxyl radicals cause the oxidation of the different substances contained in the water and ultimately lead to a mineralization and thus to a decomposition of the contaminating organic substances. In this case the ozonized olefin and the products formed in this process are initiators of a radical chain reaction that leads to a removal of the harmful substances and contaminants from the water.

The ozone for the method according to the invention is preferably produced in situ, for example by an ozone generator, the ozone being produced electrically by a plasma discharge (barrier discharge). Dry oxygen with an oxygen content of approximately 99% can be used for example as carrier gas for the ozone production. In this case the dew point of the dry oxygen is in particular below −60° C. Furthermore, it is possible for example to use a molecular sieve for the provision of oxygen, in order to obtain enriched oxygen (approximately 90%) from the ambient air. In this case the dew point of the oxygen is −20° C. or lower. In principle moist oxygen or atmospheric air can also be used as carrier gas, and in general the efficiency is greater in the event of ozone formation with dry oxygen.

Ionizing the olefins for the provision of the ozonide component for the oxidative treatment of the water can also be carried out in situ. It is also possible for ionizing the olefins to be carried out at a different location and for the resulting substance, that is to say the ozonide substance already mentioned above, to be transported to the place of consumption. This is possible since the ozonide substance is general stable and storable. The production of the component can take place in batches or continuously.

In a preferred embodiment of the method ionizing the olefins is carried out aprotically. Ozone gas that originates in particular from the ozone generator described above is used for example as carrier gas for the production process. Dry ozone gas is preferably used. The use of dry ozone gas in ionizing the olefins can be managed simply and supplies good ozonization yields. In this case the essential reaction products are the already mentioned trioxolane (ozonide) and/or tetroxane. The resulting substance is storable over a relatively long period of time. The substance is substantially insoluble in water, in particular if a high proportion of cis-ozonides is formed. In principle the ozone gas used as carrier gas can also be moist or the reaction is carried out with an aqueous solvent. Generally the dew point of the ozone gas can be between approximately −100° C. and +99° C. For a protic reaction management dry ozone gas can be conveyed for example through a washing bottle with water and is then introduced into the substance to be ozonized. With this process management hydroperoxides are predominantly formed that can also be used for the water treatment according to the invention. For the sake of simplicity, the substance formed in the ionization of the olefins is designated below somewhat simply as an ozonide substance, and this substance does not necessarily have to contain one or more ozonides. It may also be the case that organic peroxides or hydroperoxides or mixtures of peroxide and/or hydroperoxide substances and of ozonides are contained. The ozonide substance formed is present in particular as an oily or creamy substance that can be used directly for the method according to the invention.

Mono- or polyunsaturated fatty acids are advantageously used as starting substances for the ionization of the olefins. For example, oleic acid or linseed oil or mixtures of different fatty acids are particularly suitable. The fatty acids can preferably be provided in the form of vegetable oils. The oleic acid can be provided, for example, in pure form or, in a particularly preferred embodiment, as olive oil, in particular as virgin olive oil that contains approximately 70% oleic acid.

The reaction mixture in ionizing the olefins can be diluted with a suitable solvent, for example with dimethylsulfoxide (DMSO). In principle, in ionizing the olefins, water and/or for example RNH₂, ROOC and/or ROH can be used as solvents.

The aprotic ozonide formation takes place according to the so-called Criegee mechanism according to the following equation where the ozonide is produced on the double bond of the olefins (in this example oleic acid):

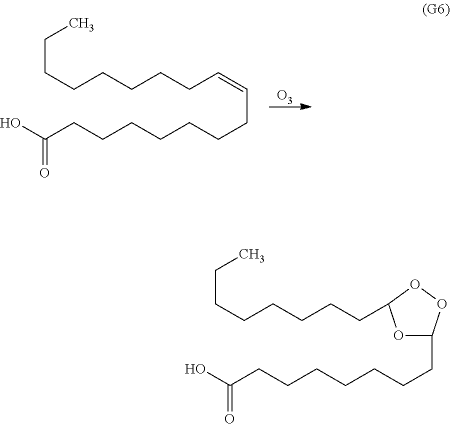

The ozonide formed in this case is very durable as a cyclic oxygen compound. It is therefore possible to store the ozonide and to use it as required. The Criegee ozonide forms a creamy or oily substance that can for example be introduced as droplets into the reaction tank or a tube reactor by a geared pump.

The dose of the component resulting from ionizing the olefins, in particular the dose of the organic ozonide substance, in the water is preferably between approximately 0.001 mL/100 m³ water and approximately 100 mL/100 m³ water. Preferably, for example, a quantity of ozonide substance is added such that the concentration in the water to be treated is approximately 1 mL ozonide substance per 100 m³ water. These details are based on the assumption of an approximately equal specific density of water and of the liquid ozonide substance, and 1 mL ozonide substance (solution) corresponds approximately to 1 g. These quantities are sufficient in order to achieve a total oxidation of the organic substances dissolved in the water.

The oxidative treatment of the water is influenced by the pH value of the water. A particularly effectively oxidative treatment can be achieved in a basic medium. Advantageously, the process according to the invention is carried out at a pH value above pH 7, in particular at pH 8 or higher. Waste water usually has a pH value greater than pH 7, so that no further measures have to be employed for the adjusting the pH value. If necessary, the pH value can be adjusted to a suitable pH value by introducing corresponding substances. In addition to the optimization of the oxidative processes, a pH value in the basic range has the further advantage that the growth of algae is inhibited in this way. In particular at a pH value above 8, the CO₂ contained in water leaves the water in gaseous form, corresponding to the lime/carbon dioxide balance in the water, so that the CO₂ necessary for assimilation is no longer available for the algae. However, experiments have shown that even at a pH value of approximately 7 the oxidative processes in the method according to the invention proceed very effectively, so that an adjusting the pH value can definitely be omitted.

The hydrogen peroxide formed during the process constitutes a further source for the hydroxyl radical formation. The oxidative effect of hydrogen peroxide in particular due to the formation of hydroxyl radicals can be enhanced by UV irradiation during the oxidative treatment.

With reference to a measurement of hydrogen peroxide in the water it is possible to assess whether the oxidative process is completed. Hydrogen peroxide can be measured when there is no longer any possibility of reaction for the hydrogen peroxide. Thus the hydrogen peroxide concentration can be used as an indicator of whether the decomposition of the trace pollutants and contaminants in the water has taken place completely.

If the hydrogen peroxide that is formed is not required after termination of the process, the excess hydrogen peroxide can then be removed. For this purpose in particular a UV treatment can be provided, for example UV irradiation with a wavelength of 254 nm. As a result the hydrogen peroxide is ultimately decomposed to water. The UV treatment can be carried out in particular at the end of the water treatment process in order to be able to provide completely uncontaminated water.

With the method according to the invention a complete oxidation (total oxidation) of the trace pollutants can be achieved without further action, so that the dissolved organic substances contained in the water to be treated are completely eliminated. Therefore the treatment process according to the invention is suitable in particular for the provision of drinking water, since it should no longer be possible to detect any dissolved organic substances (no dissolved organic carbon) in drinking water. By comparison with conventional methods, in the method according to the invention the ozone costs can be considerably reduced by comparison with dissolved organic carbon (DOC). In conventional methods the ratio M(O₃)/M(DOC) is approximately 1 (M=mass). With the method according to the invention a ratio M(O₃)/M(DOC) of approximately 0.1 can be achieved. This means that the ozone consumption can be reduced by the factor of 10. Thus the oxidative utilization of the ozone is considerably improved in the method according to the invention.

In order to be able to control the process optimally, in a preferred embodiment of the invention the process management parameters, in particular the ozone concentration and the hydrogen peroxide concentration in the water, are measured during the process. These parameters are preferably measured continuously. The metering of ozone and/or the production of ozone can be adjusted or controlled as required on the basis of these measured values. Regulation of the metering of ozone and/or the ozone production takes place particularly preferably on the basis of the measured values. The process management parameters ozone and hydrogen peroxide are measured in particular at the end of the process, for example on an overflow and/or on an outlet for the treated water. A particular advantage of the method according to the invention is that it is sufficient to control or to adjust the metering or production of only one oxidizing agent, in particular only ozone. The other oxidizing agent, specifically the component resulting from the ionization of the olefins, can be introduced so that this component is latent or present in excess in the process.

For the so-called peroxone process or also the hydrozone process (equation G3, see above) a specific ratio of added ozone and hydrogen peroxide is generally necessary, for example approximately 0.4 mg H₂O₂/L water for 1 mg ozone/L water. However, optimal metering of H₂O₂ also depends on the content of organic dissolved substances in the water, that is to say, on the so-called matrix. This content is generally not constant, so that the peroxone process is labile. The organic substance content of the water, that is to say the content of contaminating substances, can be detected using laboratory techniques, but generally in practice cannot be measured continuously, so that this value cannot be used for adjusting or controlling the peroxone process. If only the peroxone process were used for the oxidative treatment, the chain reaction with the hydroxyl radical lightweight could therefore easily come to a stop, since both agents, i.e. ozone and hydrogen peroxide, can cancel each other out without oxidation of the harmful substances being effected. However, the treatment according to the invention with an ozonide or other products from ionizing olefins provides a further source for the hydroperoxide anion that is the precursor for the hydroxyl radicals, so that the chain reaction does not come to a stop. In this case it is sufficient if the process is controlled or adjusted by metering of the ozone.

The method according to the invention for treatment of contaminated water can be used advantageously in different fields. It is generally suitable for treating waste water, and it can be used for example for waste-water purification in the agricultural field, for example for purification of waste water from greenhouses whereby in particular an oxidation and thus a decomposition of pesticides used can be achieved. In use in sewage treatment works, with the method according to the invention it is possible for example also to remove pharmacological residues. By use in the cleaning of swimming pools it is optionally possible to dispense completely with the use of chlorine chemistry. Furthermore, the method according to the invention can also be used advantageously for keeping pond areas clean or in fish farming.

In water treatment and especially in waste-water treatment exhaust gases having strong odors can be produced that are caused in particular by sulfur-containing compounds. The principle according to the invention, according to which an ozonized medium is brought into contact with a component resulting from by ionization of olefins, can also be used for treatment of exhaust gas or exhaust air. Therefore, in a preferred embodiment of the method according to the invention, in order to purify the exhaust air from the method of waste-water treatment an oxidative treatment of the exhaust air can take place, as the exhaust air is brought into contact with at least one component resulting from ionizing at least one olefin. This component is advantageously the same component also used for the actual water treatment. Therefore, with regard to further features of the component for the exhaust air treatment, reference is made to the above description. Preferably the waste air is additionally ozonized. It is preferable that the oxidative processes in treating the outgoing air proceed in a liquid phase. The chemical processes that take place are substantially the same as in the water treatment. Therefore the waste air flow is preferably moistened. This can take place particularly advantageously by gas scrubbing in the exhaust-air line that at the same time also has a further cleaning effect.

The method according to the invention for purification of exhaust gas or of exhaust air can also take place independently of the described oxidative treatment of contaminated water. The method of treating exhaust gases can be used in general for eliminating odors and for disinfection in the exhaust air treatment. For example, in industrial exhaust-gas treatment the method can be used for removing volatile organic substances (VOC). A further example of a field of use is waste collection and loading stations. Also in animal husbandry the method can be carried out, and in the inner region hygienic air conditions can be provided and moreover a purification of the exhaust air can be carried out. When used in connection with air-conditioning systems, for example, a reduction of the proportion of outside air can be achieved as an energy saving measure. The method according to the invention can be used particularly advantageously in connection with evaporative coolers whereby in particular a reduction of the chemical oxygen requirement (CSB) is achieved. Generally in this case a disinfection and sterilization is achieved, so that chemical disinfecting agents, such as for example chlorine, peracetic acid, potassium permanganate or hydrogen peroxide, may be omitted. Furthermore, contamination with Legionella or other problematic pathogens can be avoided effectively. Moreover, the growth of algae in an evaporative cooler can be combated by the exhaust-gas treatment according to the invention.

The method according to the invention is also suitable for oxidative treatment of a solid phase (solid materials). In this case the solid materials are treated, on the one hand, with ozone and, on the other hand, with at least one component resulting from the ionization of at least one olefin. Since the reactions taking place during the method according to the invention advantageously take place in an aqueous phase, a moistening of the solid materials is preferably provided. Reference is made to the above description with regard to further details relating to this method of solid material treatment. A cleaning, disinfecting or sterilizing effect can be achieved very advantageously by this oxidative treatment. The method can generally be used for cleaning and disinfecting surfaces, for example for a cleaning or preserving a tank or cleaning or preserving a wooden barrel, in particular for cleaning of wine barrels. Furthermore, the method according to the invention is suitable for processing of solid material waste, for example hospital waste, by cleaning and disinfection. Hospital waste is usually incinerated. However, since hospital waste should be assumed to include contaminants that are hazardous to health, decontaminating the waste must be carried out before the hospital waste is transported to a waste incineration plant. The method according to the invention is suitable for treating hospital waste to a particular degree, since the ozonide substance with which the solid materials are treated adheres to the solid material at least for a certain time and also still exhibits its oxidative effect even during transport. The treatment according to the invention to a certain extent exerts a depot effect, so that a longer-term decontamination is achieved and for example a repeated multiplication of pathogens during transport of the waste is avoided.

The waste is preferably comminuted, for example shredded or ground, before the oxidative treatment according to the invention or optionally also during this treatment. Due to the increase in surface area of the solid materials associated with this, the treatment according to the invention can be particularly effective.

The solid materials are advantageously moistened before or during the oxidative treatment. In particular the moistening can take place with ozonized water, for example by spraying or sprinkling, so that the moistening and the treatment with ozone can be carried out in one step.

Furthermore, the method according to the invention can be used advantageously in plant protection and in particular in treating plant pests. Experiments by the inventor have shown that a fungal attack or an insect infestation can be effectively combated by a treatment with ozone and with at least one component resulting from ionizing an olefin.

The invention also relates to an apparatus for the oxidative treatment of a liquid phase and/or a gas phase and/or a solid phase According to the invention the apparatus comprises at least one arrangement for introducing ozone and at least one arrangement for the introduction into the process of a component resulting from ozonizing at least one olefin With regard to further features of the individual elements of the apparatus, reference is made to the foregoing description.

The apparatus preferably contains a packing provided for adsorption of the component resulting from ionizing the at least one olefin.

If an exhaust-air line is present in the apparatus, a (further) arrangement for introducing an ozonide component and/or an arrangement for introducing ozone and/or an arrangement for moistening the air stream, for example a gas scrubber, can also be provided in the exhaust-air line. By this/these measure(s) the exhaust air can be additionally treated oxidatively in the manner according to the invention, independently of for example a liquid phase or a solid phase treated in the apparatus.

The apparatus according to the invention is suitable in particular for the oxidative treatment of contaminated water, for example waste water. In a first step gaseous ozone is introduced into the water, for example by a corresponding injector. The ozone is physically dissolved in the water. Next the ozonized water is brought into contact with the component resulting from the ionization of the olefins. However, this sequence of ionization and addition of this component is not obligatory and can also take place in some other way. The ozone dissolved in the water is ultimately transformed into hydroxyl radicals. In this case a radical chain reaction is initiated that causes the oxidation of the harmful substances contained in the water, so that the harmful substances are removed and mineralized. Furthermore, disinfection or sterilization of the water is achieved by the oxidative treatment.

One or more reaction tanks are provided for the reaction. Circulation preferably occurs in the reaction tank(s), so that the water can be held in the reaction tank until the process is completed, and a continuous process management is preferably achieved.

In a preferred embodiment of the apparatus according to the invention the apparatus comprises at least one tube reactor provided in the reaction tank of the apparatus. The water to be treated is conducted through this tube reactor for example by a pump. Circulation of the water is achieved by the flow conditions in and downstream of the tube reactor. The component resulting from the ionization of the olefins, or the ozonide substance, can be introduced directly into the tube reactor. In the tube reactor a packing for example of pebbles or other materials can be provided, on which the ozonide or the component is adsorbed. However, such a packing may also be provided alternatively or additionally at a different location in a tank of the apparatus or the plant. Also the metering point for the ozone gas can open into the tube reactor, preferably upstream of the optionally present metering point for the ozonide substance. In other embodiments it may be provided that ionization of the water takes place at a different location.

Ionization of the water, that is to say the dissolution of gaseous ozone in the aqueous phase, can take place for example in the tube reactor and/or at a different location in the system. The ozone is preferably introduced by one or more ozone metering points that are for example configured with a fine filter in the form of a frit and/or as an ozone injector. Since the subsequent oxidative processes generally proceed very quickly, the dissolution of the ozone in the water may be the step of the method that determines the speed. In order to further optimize the timing of the process, arrangements for improved dissolving of the ozone in the water can be provided. For example, an ozone injector can be used that according to the principle of a Venturi nozzle intensifies the mixing of the gas phase and the aqueous phase in the metering point and thus improves the dissolution of gas. Additionally or alternatively a turbulent pipe section can be provided downstream of the ozone metering point and also intensifies the dissolution of the ozone gas in the aqueous phase. Such a turbulent pipe section can for example have a length of at least 10 meters, and structures can be provided in the pipe section that produce a high Reynolds number (Re), for example Re>5000, as a measurement for the turbulence behavior.

The packing provided in a preferred embodiment of the apparatus provides surfaces for adsorption of the ozonide substance. Pebbles and/or materials consisting of metal oxides and/or of ceramic and/or glass are for example suitable for this. These materials can be contained in the tube reactor for example in the form of a packing, preferably downstream of a metering point for the ozone gas, and/or at a different location in the system. These materials or structures particularly advantageously have an oxidic surface. Thus on the one hand the structures or the packing serve(s) as an adsorptive carrier for the ozonide substance. On the other hand, ozone can also be converted on the oxidic surfaces, so that these surfaces constitute a further source for the formation of the hydroxyl radicals.

In a preferred embodiment of the apparatus according to the invention the apparatus comprises at least one UV radiation source, for example one or more UV lamps (wavelength for example 254 nm). The hydrogen peroxide contained in the water is converted to hydroxyl radicals by the UV irradiation. Thus one or more UV lamps, for example in one or more reaction tanks, provide a further source for the hydroxyl radical formation that improves the oxidative purification process. Moreover, excess hydrogen peroxide in the water can be decomposed by UV irradiation. This applies in particular when the decomposition processes are completed and there is no further possibility of reaction of the hydrogen peroxide. In order to free the water from excess hydrogen peroxide, a UV radiation source can be provided in particular in the region of an outlet of the apparatus. A plurality of UV radiation sources can also be provided at different positions in the apparatus or plant or at different process stages.

Furthermore, the apparatus according to the invention advantageously comprises at least one arrangement for measuring the ozone concentration and/or the hydrogen peroxide concentration. These process management parameters can be measured in particular continuously. On the basis of the measured values the process can be managed by a suitable control unit with or without feedback. The measurement of these parameters preferably takes place at the end of the process, for example at an overflow and/or at an outlet for the treated water. For example, the measurements, in particular the hydrogen peroxide measurement, can take place at an overflow for the treated water before the water runs into a tank in which UV irradiation takes place. The UV irradiation can take place in accordance with the measured hydrogen peroxide concentration. From the UV irradiation tank the water runs into a drainage conduit for the treated water (ultrapure water conduit). Further measurement of the parameters can be provided at this position.

Measurement of the ozone dissolved in the water can take place by various methods. A measuring method is particularly preferred in which the radiation absorption of the gas is measured and is compared with a reference absorption, and the solutions are each fed into a cell and both cells are measured alternately. Such a method is disclosed for example in German patent DE 41 19 346 [U.S. Pat. No. 5,334,536]. This method is particularly suitable, since the ozone dissolved in the water can be reliably determined even in the presence of organic substances as well as peroxidic substances. For measuring the hydrogen-peroxide content it is particularly advantageous to use an ammeter that operates in particular on the basis of three electrodes and a potentiostat.

The oxidative water treatment proceeds particularly advantageously in a basic medium. For setting an optimal pH value the apparatus according to the invention comprises an arrangement for measuring and, if applicable, for controlling the pH value with or without feedback. In this case means for setting a basic pH value are preferably provided. A pH value of 8 or higher is for example particularly advantageous. In this pH range the formation of the hydroxyl radicals necessary for the oxidative treatment takes place on a particularly large scale. Furthermore, in this pH range almost all of the carbon dioxide formed in the decomposition reactions escapes in gaseous form from the water. Studies by the inventor have for example shown that, at a pH value of 7.9 in the water after passing through the process, ozone and hydrogen peroxide can still be measured, but on the other hand at a pH value of 8.4 they can no longer be measured. A pH-measurement can take place for example at the very start of the process at the inlet of the water to be treated into the reaction tank.

The apparatus according to the invention can also comprise an ozone reducer provided for removal of excess ozone in particular from the air after completion of the process. The ozone reducer can be provided in particular for thermal destruction of the ozone. For example, for this purpose an apparatus known per se for destruction of ozone from gases with an integrated heat exchanger can be used, such as is known in particular from German patent DE 10 2004 051 945.

The metering point for the ozone gas is preferably provided upstream of the arrangement for introducing the ozonide substance. It is advantageous for carrying out the process that the ozone gas in the water to be treated is already dissolved before the water comes into contact with the ozonide substance. In particular if the water circulates in a reaction tank and/or the ozonide substance is present in adsorbed form, the metering point for the ozone gas and the metering point for the ozonide substance can also be arranged differently.

The apparatus or plant according to the invention can be combined with an exhaust air treatment system, so that disruptive and/or harmful odor emissions, which may be produced by the water treatment, can be eliminated. Such odor emissions can be produced in particular by the formation of sulfur compounds, in particular hydrogen sulfide and mercaptans (thioalcohols), or generally by bacterial decomposition products that pass over into the gas phase.

An oxidative treatment of the exhaust air or the exhaust gases from the water treatment takes place in a particularly preferred manner. In this case in principle the same chemical processes are used that are also used in the waste-water treatment, and the exhaust air is treated with a component resulting from the ionization of at least one olefin (ozonide substance). In particular when there is ozone in the exhaust air, oxidative processes that lead to an elimination of contaminants in the exhaust air are triggered along especially with the decomposition of sulfur-containing compositions to sulfur dioxide. Therefore the apparatus according to the invention advantageously has an arrangement for introducing this component into the exhaust-air line, in particular a metering point for the ozonide substance. Furthermore, an arrangement for introducing ozone into the exhaust-air line is advantageously provided. For this purpose ozone-containing exhaust air from the system may also used.

Furthermore, in a preferred embodiment an arrangement for moistening the stream of air is provided. As a result the process management in the exhaust air treatment can be configured so that the oxidative processes can proceed in an aqueous or wet phase. One or more gas scrubbers are particularly suitable as the moistening arrangement. In a gas scrubber constituents of a gas stream are washed out by a stream of washing fluid in a manner known per se. In this way on the one hand a moistening of the stream of air is achieved. On the other hand a further purifying effect is achieved. The gas scrubber(s) can preferably be operated with the water from the reaction tank(s). The stream of washing fluid and condensates from the gas scrubber can be returned to the reaction tank(s) of the waste-water treatment or can be drained off in some other way. In this embodiment of the invention, on the one hand the water can be disinfected and freed from harmful substances by the oxidative treatment. At the same time, on the other hand, compounds having strong odors that are produced in the waste-water purification are removed by gas scrubbing and where appropriate especially also by the oxidative treatment of the exhaust air. The apparatus according to the invention can therefore also be used particularly advantageously for example as a component of a sewage treatment works.

For treating the exhaust air with the component resulting from the ionization of the olefins (ozonide substance), a packing, for example a packing with pebbles, is preferably provided, on which the ozonide substance is adsorbed. The exhaust air passes through this packing. Ozone can be added by the ozone generator of the water treatment plant. In some circumstances it is possible to not add ozone from the outside if ozone-containing exhaust air from the system is used. In the plant, with appropriate metering of ozone during the water treatment, excess ozone passes over into the gas phase and can be used for this purpose. Due to the recycling of this ozone-containing exhaust air from the plant for the oxidative processes in the exhaust air treatment it may be possible to omit the ozone destroyer described above in the exhaust air treatment. Hydrogen peroxide, which may originate from the water from the reaction tank of the plant if the gas scrubber(s) is/are operated with this water, can also act additionally or alternatively as an oxidizing substance in the exhaust air treatment. In a particularly advantageous manner, therefore, the oxidative treatment pf the exhaust air can be combined with a gas scrubber operated with the hydrogen peroxide-containing water from the reaction tank(s). An alternative or additional source for hydrogen peroxide may be the packing with the adsorbed ozonide substance in the exhaust gas system. Thus, it is not necessary to add external hydrogen peroxide. The oxidative processes in the exhaust air preferably proceed in a basic medium. Since the water from the reaction tank(s) preferably already has a basic pH value, adding potentially environmentally harmful basic reagents, such as for example sodium hydroxide solution, for adjusting the pH value is not necessary.

The thioalcohol compounds and hydrogen sulfide in the exhaust air can for example be oxidized to sulfur dioxide by the initiated oxidative processes in the exhaust air. Sulfur dioxide is water-soluble and can be washed out with, where applicable, an additional gas scrubber. For example, in the flow direction of the exhaust air it is possible to provide a first gas scrubber, then a packing for absorption of the ozonide substance and subsequently a further gas scrubber. Upstream of the packing the ozonide substance can be metered in in liquid form or in the form of gas or vapor. If gaseous ozonide is used, the ozonide gas used may be that resulting from the production of the paste-like ozonide substance for the water treatment. Embodiments are also possible with only one gas scrubber upstream or downstream of any packing for absorption of the ozonide substance. In particular at high hydrogen sulfide concentrations in the exhaust air, for example at 1000 ppm or more, two gas scrubbers are advantageous that are provided upstream and/or downstream of the packing for adsorption of the ozonide substance. The acidic condensate from the gas scrubber(s) can be used for adjusting the pH value in the outlet or, if applicable, additionally or alternatively in the inlet of the water treatment plant.

Particularly high hydrogen sulfide concentrations are produced for example in the event of digestion of the sewage sludge. For oxidative decomposition of highly odorous compounds of the exhaust gases produced in this case, high oxidation capacities are necessary that are achieved in particular by the combination according to the invention of adding ozone with the further oxidizing component (ozonide substance or ozonide), preferably in combination with gas scrubbing. The addition of ozone can be regulated for example by a measurement of the hydrogen sulfide concentration at the exhaust gas outlet.

This exhaust-gas treatment apparatus according to the invention is not limited to those installations that carry out a water treatment on the basis of ozone and an ozonide substance according to the above description. On the contrary, this oxidative exhaust-gas treatment can also be used for other exhaust gases or exhaust air, and the arrangement for introducing ozone is an arrangement for ozonizing the exhaust gases or the exhaust air. The invention therefore also comprises an apparatus for purification of exhaust gases independently of the waste-water treatment according to the invention. In particular this exhaust gas or exhaust air treatment is generally suitable for the exhaust air treatment in sewage treatment works. With the apparatus it is possible above all to remove sulfur-containing compounds as highly odorous emissions from the air (exhaust air). The oxidative and purifying processes in the exhaust air are triggered by a treatment with an oxidizing component resulting from ionizing at least one olefin, in particular in the presence of ozone as a further oxidizing agent. The component on the basis of ozonized olefins is preferably present in adsorbed form on a packing through which the exhaust air passes. The packing can consist for example of pebbles or other materials. The chemical processes triggered hereby correspond predominantly to the processes that also operate in the water treatment according to the invention. In this respect reference is made to the above description relating to waste-water treatment with regard to further details and in particular with regard to the oxidizing component.

The process management can be configured so that ozone in the exhaust-air line is metered in. If applicable, ozone-containing exhaust air from an entire installation can also be used therefor. The component on the basis of the ozonized olefins can be metered-in in liquid or gaseous form. Furthermore, the oxidative processes can be intensified by the presence of hydrogen peroxide. Hydrogen peroxide is preferably provided by hydrogen peroxide-containing liquids, in particular from water, originating from the entire system. Particularly advantageously, at least one arrangement for moistening the stream of air is provided whereby therefor in particular one or more gas scrubbers can be used for this. Water, in particular hydrogen peroxide-containing water, from the entire system, can be used as washing fluid for the gas scrubbing. In principle the exhaust air treatment according to the invention is based on moist ozone gas being passed over an ozonide surface or another peroxidic surface, and the moistening of the ozone gas is preferably achieved by a gas scrubber.

Furthermore, the apparatus according to the invention is suitable for oxidative treatment of solid materials, for example for treatment of barrels, for example wooden barrels, or of tanks or for treatment of waste. For introducing ozone and for introducing the ozonide substance, a common arrangement or separate arrangements, for example two different metering points, can be provided. Furthermore, a moistening arrangement, for example a sprinkler arrangement, can be provided, by which water, which may optionally be ozonized, is introduced. In this case this sprinkler arrangement may also constitute the arrangement for introducing ozone. Furthermore, depending on the type of solid materials or surfaces to be treated, a comminuting apparatus can be provided, for example for treating waste. In treating barrels or tanks with such an apparatus, above all disinfection of the inner surfaces of the barrels or tanks is achieved. In an apparatus for treatment of barrels or tanks it is possible for example to arrange a spray head advantageously sealed at the inlet point into the barrels or the tank, in the interior of the barrel or of the tank. By means of the spray head introducing ozone and of the ozonide substance can take place simultaneously or at different times. Furthermore, an extractor apparatus for final removal of liquids from the barrel or the tank can be provided.

Finally, according to the invention the apparatus comprises an evaporative cooler, with which is associated at least one arrangement for introducing ozone into the evaporative cooler and for the introduction into the evaporative cooler of a component resulting from ionizing at least one olefin (ozonide substance). For introducing ozone and for introducing the ozonide substance, a common arrangement or separate arrangements, for example two different metering points, can be provided. In the case of evaporative coolers, which are used for example in the context of air-conditioning systems, contamination is a widespread problem. In particular, if a packing is used to increase the surface area in the evaporative cooler, the danger of contamination is great. The use of the method described above for such evaporative coolers, that is to say the combined treatment of the evaporative cooler with ozone and with the ozonide substance, has the advantage that effective sanitization of the evaporative cooler is achieved by the oxidative processes being performed, and a contamination with for example algae and bacteria is reliably avoided. At the same time the triggered oxidative chain reactions are so effective that the ozone used is completely converted and therefore no potentially harmful ozone emissions occur.

Further features and advantages of the invention are disclosed by the following description of embodiments in connection with the drawings. In this case the individual features can be implemented in each case separately or in combination with one another.

In the drawings:

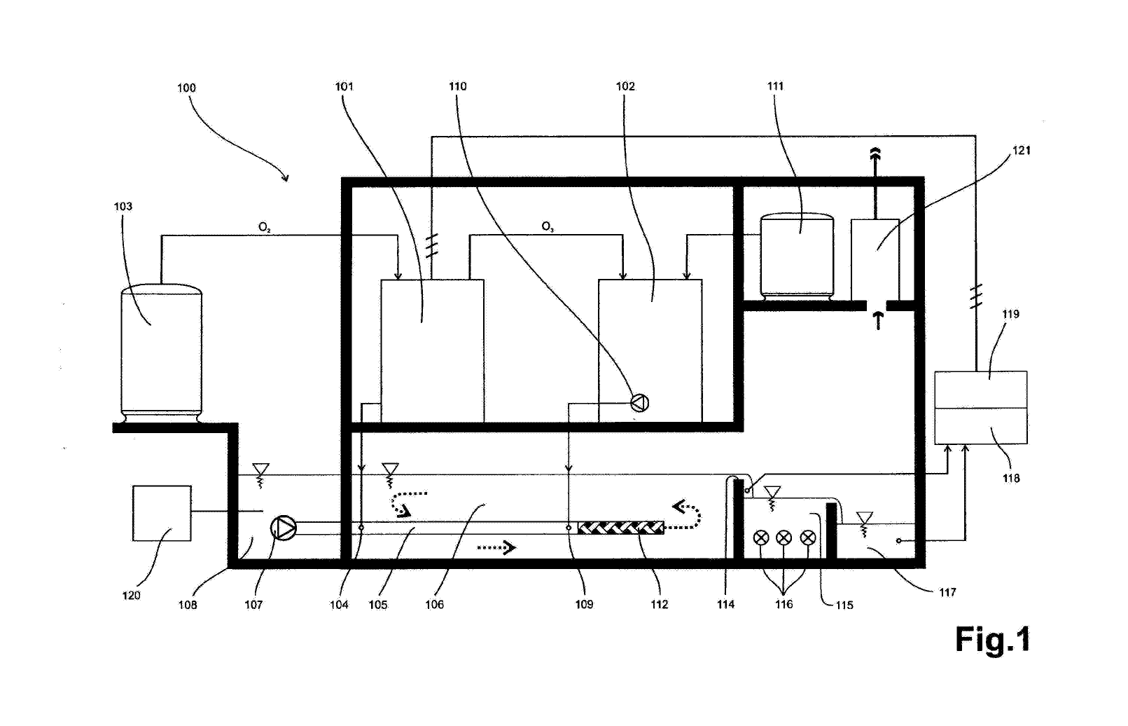

FIG. 1 shows a first embodiment of an apparatus according to the invention for the oxidative treatment of contaminated water;

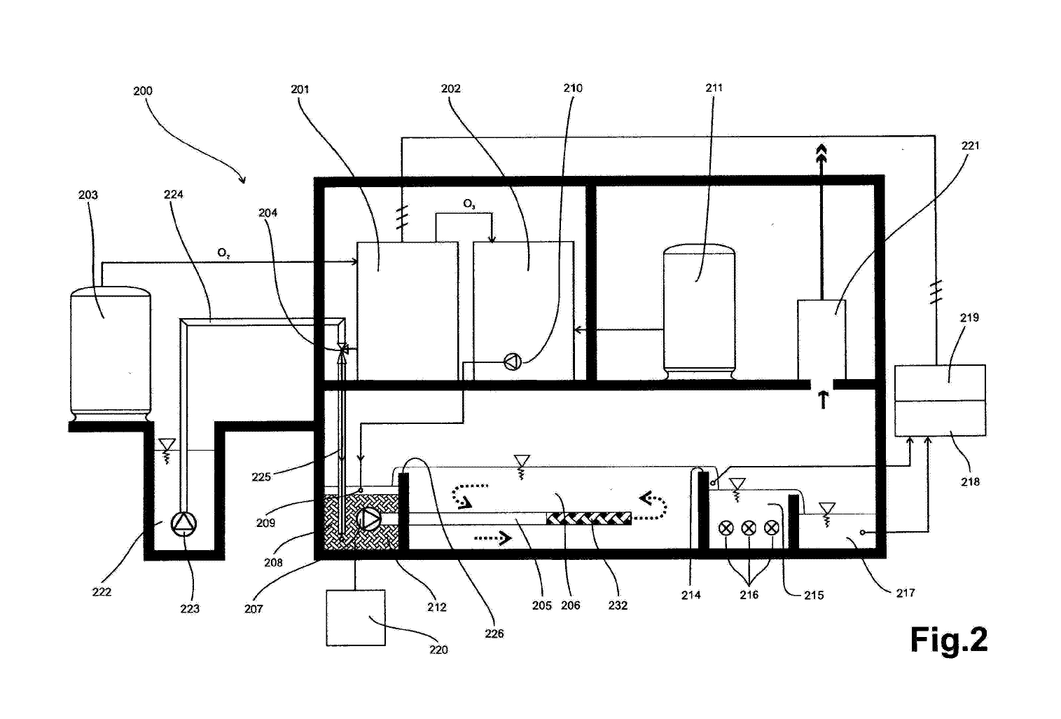

FIG. 2 shows a second embodiment of an apparatus according to the invention for the oxidative treatment of contaminated water;

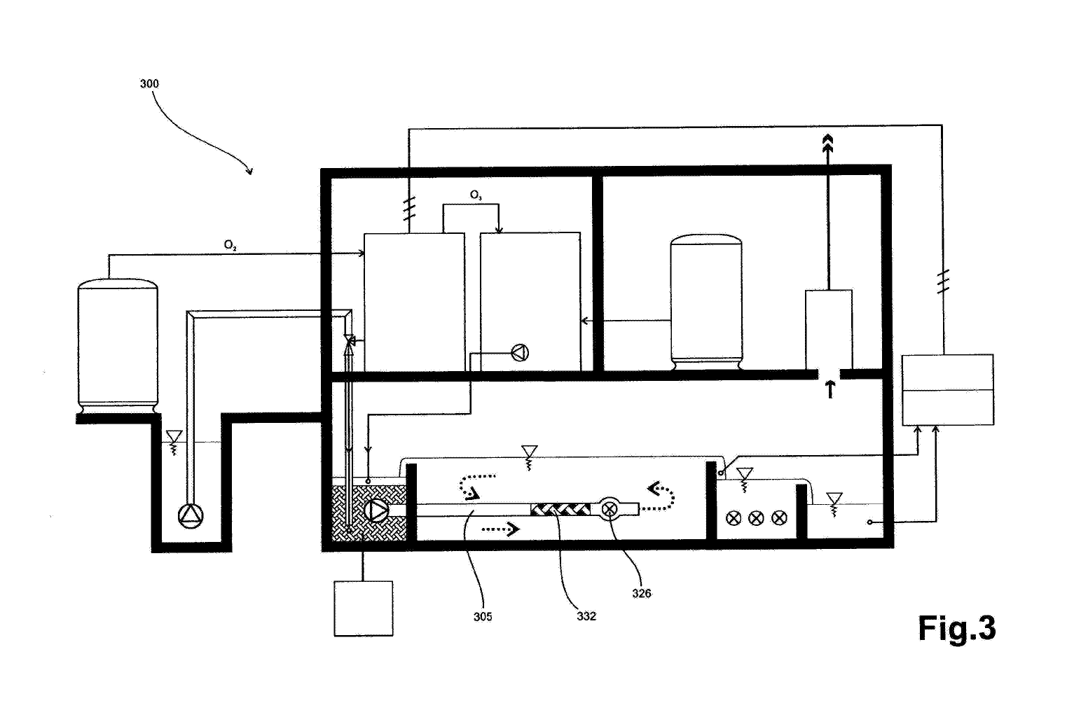

FIG. 3 shows a third embodiment of an apparatus according to the invention for the oxidative treatment of contaminated water;

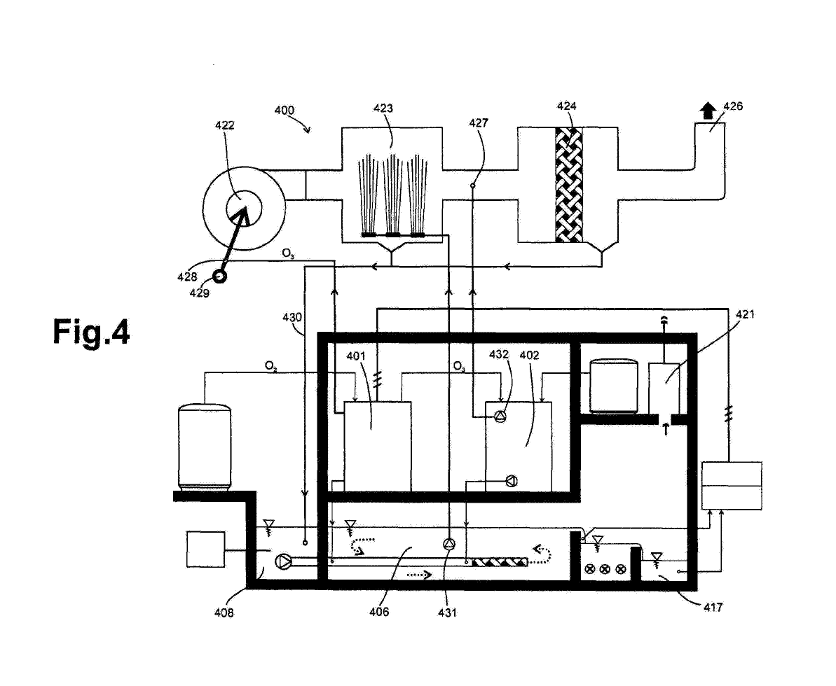

FIG. 4 shows a fourth embodiment of an apparatus according to the invention for the oxidative treatment of contaminated water with additional exhaust air treatment;

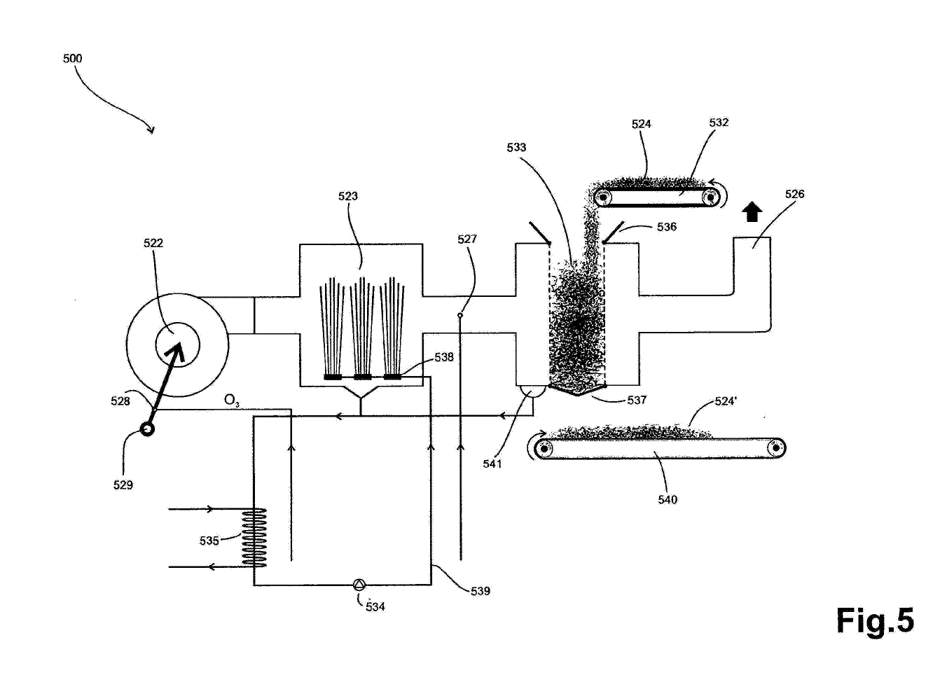

FIG. 5 shows an embodiment of an apparatus according to the invention for the oxidative treatment of comminuted solid materials;

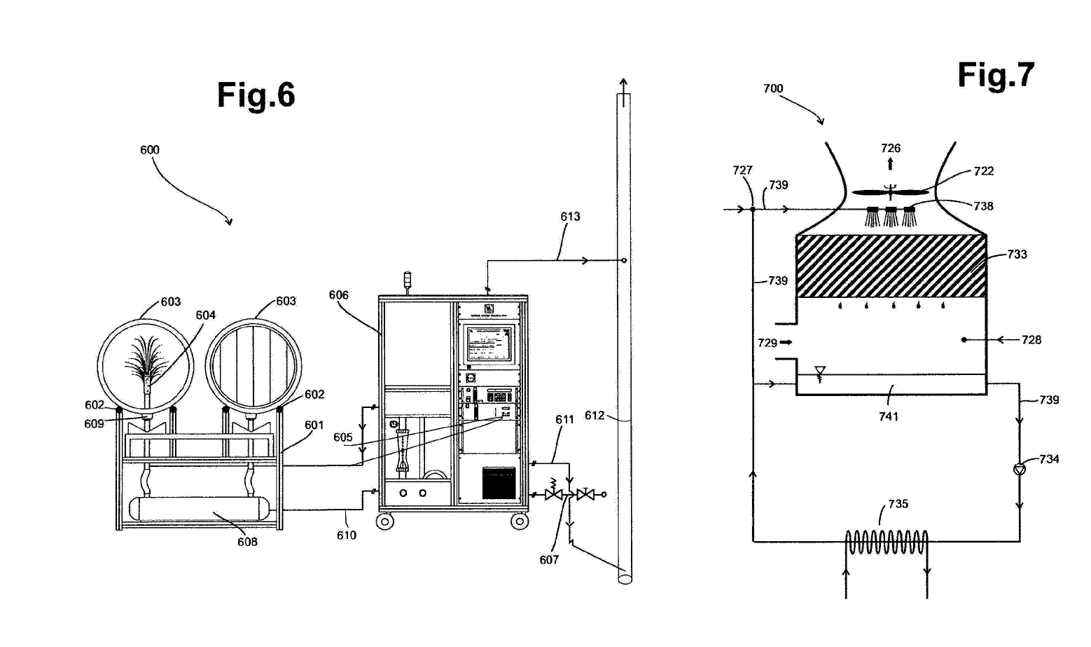

FIG. 6 shows an embodiment of an apparatus according to the invention for the oxidative treatment of barrels or tanks; and

FIG. 7 shows an embodiment of an evaporative cooler according to the invention.

FIG. 1 shows a first embodiment of an apparatus 100 according to the invention for the oxidative treatment of contaminated water. Essential components of the apparatus are an ozone generator 101 and a unit 102 by which an ozonide substance, in particular organic ozonides (and/or peroxides and/or hydroperoxides), are provided. A tank 103 stores dry oxygen introduced into the ozone generator 101. The oxygen serves as carrier gas for the ozone formation in the ozone generator 101, and the ozone is produced in particular electrically by plasma discharge in a manner known per se. The ozone formed is introduced into a tube reactor 105 by an ozone metering point 104 preferably equipped with a fine filter (frit). The tube reactor 105 is located in a reaction tank 106. The water to be treated is conveyed by a pump 107 into water inlet tanks 108 and introduced into the tube reactor 105. The water to be treated runs through the tube reactor 105 and is admixed with ozone in the metering point 104. The gaseous ozone is dissolved in the aqueous phase. Turbulent flow conditions that promote efficient dissolving of gas preferably prevail in the tube reactor 105. In this case the ozone can react directly for example with aliphatic harmful substances as well as with bacterial substances, in particular with proteins and lipids, and produce a first oxidative treatment stage. Further along the tube reactor 105 is located a metering point 109 for the organic ozonide or the ozonide substance. The ozonide arrives by a pump 110, for example a geared pump, at the metering point 109 and is for example added drop by drop.

In this example the starting material for the ozonide production in the unit 102 is a mono- or polyunsaturated fatty acid, in particular oleic acid provided in the form of virgin olive oil in the storage container 111. The ozonide forms in the unit 102 aprotically according to the Criegee mechanism, and dry ozone gas from the ozone generator 101 serves as carrier gas. The ozonide (ozonized olive oil) forms a pasty or creamy substance introduced into the tube reactor 105 by the geared pump 110. In other embodiments it may be provided that the ozonide is produced at another location and as a prepared ozonide substance is transported to the installation and is used there.

For the ozonide production the ozone gas is introduced into the substance to be ozonized, that is to say for example into the virgin olive oil. In this case the concentration of the ozone gas may be for example 120 g O₃/m³ O₂. Depending on the layout of the installation the ozonide production may be completed after approximately 2 to 8 hours. The successful ozonization process can be monitored in particular with reference to the consistency and the coloring of the resulting compound that in the course of the ozonization becomes increasingly pasty and whitish. The resulting ozonide has a very high content of peroxide compounds. The peroxide value can be for example between approximately 300 and 1000 or higher, in particular up to 6000.

Downstream of the metering point 109, in the tube reactor 105 there is a region 112 that contains a packing on which the ozonide substance, which is only just water-soluble, is adsorbed. This packing preferably consists of pebbles or other structures, for example consisting of metal oxide or ceramic materials or of glass that are suitable for adsorption of the ozonide substance. Due to the adsorption of the ozonide substance there are always traces of ozonide or other peroxide compounds available for the oxidative reactions. Particularly advantageously, the adsorption materials have oxidic surfaces on which a further conversion of the ozone dissolved in the water takes place. Metal oxides or ceramics are suitable in particular for process management at pH values above 7, and for example aluminum oxide or manganese oxide can be used. Further suitable materials are for example silicon oxide (silica gel) or porous glass.

The high outflow speed from the tube reactor 105 leads to a circulating movement in the reaction tank 106, indicated here by broken-line arrows. Due to the delivery capacity of the pump 107 an overflow is produced in the region 114, and the water is directed into an overflow tank 115. An arrangement 116 for UV irradiation of the water is provided in the overflow tank 115. In this region excess hydrogen peroxide is destroyed by the UV irradiation. The overflowing ultrapure water enters the ultrapure water conduit 117 as the outlet of the apparatus 100. The water in the ultrapure water conduit 117 is continuously measured with regard to ozone and hydrogen-peroxide concentration. For this purpose a sensor 118 for hydrogen peroxide and a sensor 119 for ozone are provided. A further hydrogen peroxide measurement can also take place in particular in the overflow 114 between the reaction tank 106 and the overflow tank 115. On the basis of the process parameters recorded by these measurements, control and/or regulation of the metering of ozone and/or ozone production take place in the ozone generator 101. For this purpose a control and/or regulating unit not illustrated in greater detail is provided, so that the ozone production and/or the metering of ozone can be regulated or controlled according to consumption. Excess ozone gas emanating from the water can be thermally eliminated by an ozone destroyer 121 provided above the ultrapure water conduit 117.

A pH value measuring unit 120 is provided at the water inlet 108. This unit records the pH value of the introduced water. Since the method according to the invention is preferably carried out in a basic environment, in particular at a pH value of 7 or higher, in particular at pH 8 or higher, the pH value is optionally set to a suitable pH value.

FIG. 2 illustrates a further embodiment of an apparatus 200 for oxidative water treatment. An ozone generator 201 and a unit 202 for providing the ozonide substance are provided in a manner comparable to the embodiment according to FIG. 1. Dry oxygen stored in the container 203 is used as carrier gas for the ozone production in the ozone generator 201. Also in this example ionizing of unsaturated fatty acids takes place in the unit 202 using oleic acid held ready in the storage container 211 in the form of virgin olive oil.

In this embodiment, particular emphasis is placed on the water treatment in the direct ozone reaction that in particular also effects disinfection or sterilization of the water to be treated. This embodiment therefore differs from the example illustrated in FIG. 1 by the measures for introducing the ozone gas into the water. The water to be treated with the harmful substances it contains is conveyed from a well 222 by a feed pump 223 and reaches a metering point 204 for the ozone by a pipeline 224. In this embodiment the metering point 204 is configured as an injector located above the water level of the reaction tank 206. The injector 204 is designed in the form of a Venturi nozzle, so that at the injector outlet a counter-pressure is generated that produces an increase in the water solubility of the ozone gas. This is followed by a turbulent pipe section 225 that opens into the inlet tank 208. In this case the turbulent pipe section 225 is for example at least 10 m long. This pipe section 225 is configured so that substantial turbulence is produced (Reynolds number>5000). Due to this measure optimal dissolution of the ozone gas in the water is achieved. Measurements have shown for example that the gas bubbles that rise up in the inlet tank 208 are ozone-free.

A unit 220 for measuring and if applicable regulating the pH value is located in the inlet tank 208. The pH value is preferably set in a basic range. A unit 212 for measuring and, if applicable, regulating the pH value is located in the inlet tank 208. The packing 212 consists for example of a pebble bed or of metal oxide or ceramic materials. The packing preferably has oxidic surfaces, so that here too conversion of the ozone can take place. The metering point 209 for the ozonide substance is located in the inlet tank 208. The ozonide substance is for example introduced as an oily or creamy substance into the tank 208 by a geared pump 210. The ozonide substance is virtually water-insoluble and is adsorbed on the packing 212. A circulation pump 207 that draws the water through the packing 212 and feeds it into a tube reactor 205 is located in the inlet tank 208. In this case the pumped volume of the circulation pump 207 is greater than the pump volume of the feed pump 223. A further region with a packing 232 for the adsorption of the ozonide substance is provided in the downstream region of the tube reactor 205. Due to the high outflow speed from the tube reactor 205, a circulating movement takes place in the reaction tank 206 so that the substances (contaminants) contained in the water can be completely oxidized and decomposed.

An overflow 214 for the treated water is provided and opens into an overflow tank 215, in a comparable manner to the embodiment of the apparatus according to FIG. 1. A further overflow 226 for the water from the reaction tank 206 is located between the reaction tank 206 and the inlet tank 208, so that the overflowing water passes through the packing 212 a number of times.

An excess of hydrogen peroxide can occur in the reaction tank 206 due to the oxidative processes being performed. The hydrogen peroxide promotes the decomposition processes and is therefore advantageous. The hydrogen peroxide can be recirculated through the overflow 226, so that it is uniformly available. In this case it is particularly advantageous that no external hydrogen peroxide has to be metered in, so that the potentially dangerous storage and metering of hydrogen peroxide is omitted in an installation according to the invention. If the hydrogen peroxide concentration is too high, the metering of ozone can be throttled.

An arrangement 216 for UV irradiation of the water is provided in the overflow tank 215, so that excess hydrogen peroxide can be decomposed before it leaves the installation. Downstream of the overflow tank 215 the water then reaches the ultrapure water conduit 217 by a further overflow. Here and, if applicable, in the overflow 214 a hydrogen peroxide measurement 218 and an ozone measurement 219 can take place. The measured values are used for controlling and/or regulating the addition of ozone and/or the ozone production. The measured values of the hydrogen peroxide concentration at the overflow 214 can also be used for controlling the UV treatment in the tank 215. Excess ozone gas emanating from the water can be eliminated by an ozone destroyer 221.

FIG. 3 shows a further example for a preferred embodiment of the apparatus 300 according to the invention for the oxidative treatment of contaminated water. The apparatus 300 is substantially comparable to the embodiment 200 that has been explained with reference to FIG. 2. In contrast, the embodiment 300 has a further UV radiation source 326 in the downstream region of the tube reactor 305. The UV lamp 326 is located downstream of the packing 332 provided for adsorption of the ozonide substance. Due to the UV irradiation in the tube reactor the formation of hydroxyl radicals from hydrogen peroxide is further intensified, so that due to the UV irradiation at this position the oxidative decomposition of harmful substances is further improved.

With the method according to the invention, which can be carried out in particular by the described apparatuses, above all organic contaminated water can be processed very effectively. Compared to an ozone treatment, as is known per se, the method according to the invention, in which the additional oxidizing component on the basis of ozonized fatty acid is used, enables a substantially more extensive decomposition and a mineralization of the organic substances contained in the water, and in particular also a decomposition of halogenated organic hydrocarbons and other trace pollutants. The oxidative decomposition or purification process according to the invention is based on a radical chain reaction, and hydroxyl radicals are the crucial molecules. Due to the combination of ionizing the water with a treatment with the component on the basis of ozonized fatty acids different sources are available for the hydroxyl radical formation, which influence each other, so that precisely this combination causes the very effective oxidative treatment. In this case, in particular, organic ozonides are the initiators of the radical chain reaction. On contact with the water, the substantially water-insoluble ozonide decomposes into the aldehyde and carboxylic acid fractions, and hydrogen peroxide is formed (equation G8). In the presence of ozone, ultimately the hydroxyl radical forms according to the following equations, and the equation G7 describes the reaction constant for the reaction of the anion with ozone in order to form the hydroxyl radical dependent upon the pH value and the pKa value.

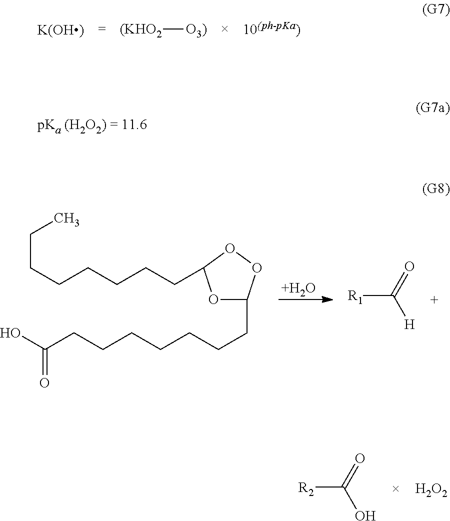

Thus, for the formation of the hydroperoxide anion as precursor of the hydroxyl radical, to start with there are two sources, namely from the decomposition of ozone (equation G1, see above) and from the decomposition of the ozonide (equation G8), in particular at basic pH values. Also the carboxylic acid produced from the organic ozonide can also, as intermediary in the entire system, is part of the radical chain reaction (ROO.) and can constitute a further source for the hydroxyl radical-formation. The oxidic surfaces of the packing materials at which ozone can be converted can act in the apparatus according to the invention as a further source for the hydroxyl radical formation. A further source for the hydroxyl radical formation can be achieved by the UV irradiation in the reaction tank, and hydrogen peroxide is converted to the hydroxyl radical by UV action.

Measurements show that after treatment with the ozonide substance according to the invention no more ozone can be detected in the treated water. Hydrogen peroxide can only be measured when there is no longer any possibility of reaction of the hydrogen peroxide, in other words when the organic substances contained in the water are completely decomposed. This is the case above all at a pH value of 8.4 or more. At pH values below 8, for example at pH 7.9, it may still be possible to measure ozone and hydrogen peroxide. The effect of the pKₐ value (equation G7a) on the stability of the ozone vanishes due to the presence of ozonide. Over-metering of ozone in the presence of the aprotically produced ozonide then leads to a rise in the hydrogen peroxide content. This excess hydrogen peroxide can be eliminated, if required, by UV irradiation.

FIG. 4 illustrates a further configuration of the apparatus 400 according to the invention, in which in addition to the oxidative water treatment a more far-reaching treatment of the exhaust air from the system is provided. FIG. 4 shows in the lower part the apparatus for the actual water treatment that corresponds substantially to the embodiment shown in FIG. 1. The exhaust-air line of the installation is shown in the upper part of the illustration. During the waste-water treatment odor emissions are often produced that are attributable in particular to sulfur compounds (for example hydrogen sulfide, mercaptans). According to the invention these highly odorous compounds are eliminated by gas scrubbing and/or by an oxidative treatment in the exhaust-air line. The exhaust air from the water treatment, illustrated here by the arrow 429, is fed into the following exhaust gas system by a fan 422. First of all the exhaust gas runs through a gas scrubber 423. The gas scrubber 423 is supplied by a pump 431 with water from the reaction tank 406 that serves as the washing fluid in the gas scrubber 423 This water contains hydrogen peroxide as a result of the oxidative processes during the water treatment. The hydrogen peroxide that enters the exhaust-air line in this way promotes the oxidative processes during the exhaust-gas treatment. After the gas scrubbing the exhaust gas enters a region with a packing 424 provided for the absorption of ozonide and/or other peroxide compositions that are supplied by ozonizing unsaturated fatty acids. This oxidizing component is introduced in liquid or in gaseous form into the exhaust-air line via the metering point 427. This oxidizing component is conveyed, for example in the form of an ozonide-containing gas, by a gas pump 432 out of the unit 402 provided for supplying the ozonide substance for the water treatment. Liquids and condensates from the gas scrubber 423 and from the region with the packing 424 run via a drainage line 430 back into the tank(s) of the waste-water treatment system. In this example the liquids run back into the water inlet tank 408. Due to the products formed during the oxidative treatment, in particular SO₂, the condensate is generally acidic, depending upon the contamination of the exhaust air. Therefore, it can also be used for adjusting the pH value in the installation. For example, the pH value of the water running out can be adjusted or neutralized by the condensate, by introduction of the condensate into the ultrapure water conduit 417. If necessary, the SO₂ formed during the oxidative treatment can be washed out by a further gas scrubber. The purified exhaust air can be released by the outlet 426.

Ozone can be metered into the exhaust-air line by the ozone metering point 428 in the fan 422. This ozone originates from the ozone generator 401 of the water treatment plant. Alternatively or additionally, for example, the ozone-containing exhaust air from the ozone destroyer 421 can be fed into the exhaust gas system. In such an embodiment the ozone destroyer 421 may possibly be omitted. In principle the basis for the exhaust air treatment according to the invention is that the exhaust gas in the form of moist ozone gas passes through a surface with ozonide and/or other peroxide compounds. In this case the moistening of the exhaust gas enriched with ozone is preferably resulting from gas scrubbing, and the gas scrubber is operated with peroxide-containing water from the water treatment.

FIG. 5 shows an embodiment of an apparatus 500 for treatment according to the invention of solid materials that are comminuted in this example. These materials may be in particular shredded waste, for example hospital waste. Comminuted waste 524 is introduced into a treatment container 533 by a conveyor belt 532. The inlet and the outlet of the treatment container 533 can be closed by respective valves 536 and 537. A moistened stream of air enriched with ozone and with ozonide flows through the treatment container 533. For this purpose an ozone metering point 528 is provided in the air supply 529 to the apparatus 500, and the inflowing air is enriched with ozone. The air 529 supplied to the apparatus is propelled by a fan 522. The stream of air runs through a moistening unit 523. Water nozzles 538 that atomize water are provided in the moistening unit 523. The water nozzles 538 are fed by a water system 539 supplied by a pump 534. The stream of air enriched with ozone and moistened then passes a metering point 527 for ozonide. In this case the air stream is additionally treated with ozone or generally with at least one component resulting from ionizing at least one olefin. This oxidizing component can be introduced in liquid or in gaseous form. Then the stream of air enters the treatment container 533 with the waste to be treated. The accompanying ozonide component is adsorbed on the solid materials and the described oxidative processes can proceed in the prevailing moist medium. The waste 524′ treated in this way is ejected by the valves 537 at the outlet of the container 533 and can be transported away by a conveyor belt 540. Excess liquid in the treatment container 533 collects at a water outlet 541 of the treatment container 533 and is returned to the water system 539. After running through the container 533 the air leaves the apparatus through an exhaust-air opening 526.

The water in the water system 539 cools due to the evaporation of the water in the moistening unit 523. This cooling by evaporation can be used with a heat exchanger 535 in order to cool other media (indirect evaporative cooler). Furthermore, the heating of the water that occurs in this case in the water system 539 is advantageous for the effective moistening of the stream of air in the moistening unit 523.

As an alternative to the treatment container 533 shown in FIG. 5 and the conveyor belts 532 and 540, continuous operation with a cyclone is for example also possible in a comparable manner.

FIG. 6 illustrates an apparatus 600 according to the invention for oxidative (sterilizing) treatment of the inner surfaces of wine barrels 603 or of tanks on the basis of the method according to the invention. The barrels 603 or tanks to be cleaned are supported on a storage rack 601 with positioning rollers 602. A spray head 604 projects into the interior of the barrels 603 through a corresponding opening in the barrels 603 or tanks. In the course of the treatment an ozonide substance is sprayed by the spray head 604 into the barrel 603 or tank. Then, simultaneously or beforehand, ozone water, that is to say water admixed with ozone, or ozone gas is introduced via the spray head 604 into the interior of the barrel 603 or of the tank. For this purpose the spray head 604 is connected by a hose connection 605 as supply conduit with a supply unit 606. By means of the supply unit 606 the provision and the delivery of ozonized water (or ozone gas) and the ozonide substance take place via the hose connection 605. Fresh water is supplied to the supply unit 606 by a fresh water supply 607. A collecting tank 608 for liquids from the barrels 603 or tanks is provided in the lower region of the storage rack 601. As soon as the oxidative processes within the barrels 603 or tanks are concluded, the liquid contained in the barrels 603 or tanks can be drawn off by a suction extraction element 609 and collected in the collecting tank 608. The liquid from the collecting tank 608 is directed by a hose connection 610 as discharge conduit into the supply unit 606 and from there is discharged via an outlet 611 into a discharge conduit 612. The resulting exhaust gases can also be discharged by a ventilation conduit 613.

FIG. 7 shows an evaporative cooler 700 according to the invention provided for cooling a stream of air. This evaporative cooler may for example be a component of an air-conditioning system. The stream of air is indicated by arrows 729 and 726, and the air enters the evaporative cooler 700 from an air supply 729 and leaves the evaporative cooler 700 by the air outlet 726. In this case the air runs through a packing 733, for example a plastic packing. The packing 733 is sprayed with water by a plurality of nozzles 738, so that the packing 733 is wetted with moisture. Due to the evaporation of the water in particular in the packing 733, a cooling effect is achieved for the air flowing through the packing 733. The evaporation of the water can be supported by a fan 722. In the lower region of the evaporative cooler is a water container 741 for collecting excess liquid. The water container 741 is part of a water system 739 supplied by a pump 734 and feeds the nozzles 738. Due to the evaporation of water in the packing 733, that is to say within the water system 739, the circulating water cools and can be used for cooling purposes for a heat exchanger 735. For the oxidative treatment of the evaporative cooler 700 according to the invention, on the one hand an ozone metering point 728 is provided for feeding ozone gas or ozonized water into the stream of air. On the other hand an ozonide metering point 727 is provided for the substance formed by ionizing at least one olefin, that is to say for the ozonide substance, and in the embodiment illustrated here the ozonide substance is fed into the water system and is distributed on the packing 733 by the nozzles 738. The combined treatment with ozone and ozonide leads to the described oxidative processes and chain reactions and thus to sanitization of the evaporative cooler 700, so that bacterial contamination and algae growth in particular in the packing 733 is reliably avoided and a clean and simultaneously cooled stream of air is achieved.

1. A method of oxidative treatment of a liquid, gas or solid phase, the method comprising the step of:
using ozone and at least one organic ozonide for the treatment.

2. The method according to claim 1, wherein the method is carried out in the presence of water.

3. (canceled)

4. The method according to claim 1, further comprising the step of:
adsorbing the organic ozonide with a packing.

5. The method according to wherein the liquid phase is contaminated water, the method further comprising the step of:
ionizing the water and
treating the ionized water with the at least one organic ozonide.

6. The method according to claim 5, further comprising the step, for the treatment of the exhaust air from the method, of
treating the exhaust air with at least one organic ozonide, and wherein the exhaust air is preferably ozonized.

7. The method according to claim 1, wherein the gas phase is an exhaust gas.

8. The method according to claim 1, wherein the solid phase is formed by inner surfaces of wooden barrels or tanks.

9. The method according to claim 1, the solid phase is formed by waste, the method further comprising the step of:
comminuting the waste before the oxidative treatment.

10. An apparatus for oxidative treatment of a liquid, a gas or a solid phase, the apparatus comprising:
means for introducing ozone; and
means for introducing an organic ozonide.

11. The apparatus according to claim 10, further comprising:
a packing for adsorption of the component resulting from ozonizing the at least one olefin and comprised of pebbles or materials consisting of metal oxides or ceramic or glass, and having oxidic surfaces.

12. The apparatus according to claim 10, at least one wherein the means for introducing a organic ozonide is provided in an exhaust-air line of the apparatus.

13. The apparatus according to claim 10, further comprising:
means for introducing ozone or means for moistening the stream of air in an exhaust-air line of the apparatus.

14. The apparatus according to claim 10, wherein the apparatus is configured for oxidative treatment of contaminated water, and the means for introducing ozone is an arrangement for ozonizing the water.

15. The apparatus according to claim 10, wherein the apparatus is configured for oxidative treatment of exhaust gases or exhaust air, and the means for introducing ozone is an arrangement for ozonizing the exhaust gases or the exhaust air.

16. The apparatus according to claim 10, wherein the apparatus is configured for oxidative treatment of solid materials, and further comprises:
means for moistening the solid materials.

17. The apparatus according to claim 16, wherein the apparatus is configured for oxidative treatment of inner surfaces of wooden barrels, or tanks.

18. The apparatus according to claim 16, wherein the apparatus is configured for oxidative treatment of waste, and further comprises:
means for comminuting the waste before or during the oxidative treatment.

19. An evaporative cooler further comprising:
means for introducing ozone, and
means for introducing a organic ozonide, into the evaporative cooler.
